(19) United States
(12) Patent Application Publication
(10) Pub. No.: US 2016/0144155 A1
(43) Pub. Date: May 26, 2016

(54) OCCLUSION SHEATH FOR IMAGING CATHETER

(71) Applicant: AVINGER, INC., Redwood City, CA (US)

(72) Inventors: John B. SIMPSON, Woodside, CA (US); Theodore W. KETAI, San Francisco, CA (US); Mark W. ASKEW, San Francisco, CA (US); Serena S. LOH, San Carlos, CA (US); Guip T. NGUYEN, San Jose, CA (US); Stephen C. DAVIES, El Dorado Hills, CA (US); Richard R. NEWHAUSER, Redwood City, CA (US); Peter H. SMITH, Pacifica, CA (US)

(21) Appl. No.: 14/899,877

(22) PCT Filed: Jul. 1, 2014
(86) PCT No.: PCT/US14/45132

§ 371 Date: Dec. 18, 2015

Related U.S. Application Data
(60) Provisional application No. 61/841,843, filed on Jul. 1, 2013.

(51) Int. Cl. A61M 25/10; A61B 17/3207; A61B 5/00

(52) U.S. Cl. CPC A61M 25/10; A61B 5/0066; A61B 17/3207; A61M 2025/1052; A61M 2025/1079; A61M 2025/1081; A61M 2025/109

(57) ABSTRACT

An occlusion sheath having an expandable member is described herein. The occlusion sheath is configured to occlude or limit blood flow to a vessel while providing a central lumen to allow a catheter, such as an imaging catheter and/or an atherectomy catheter, to travel therethrough.

### CROSS REFERENCE TO RELATED APPLICATIONS

This application claims priority to U.S. Provisional Patent Application No. 61/841,843, titled “OCCLUSION SHEATH FOR IMAGING CATHETER,” and filed Jul. 1, 2013, the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death in the United States while PAD is the leading cause of amputation in patients over 50 and is responsible for approximately 160,000 amputations in the United States each year.

Coronary artery disease (CAD) and Peripheral artery disease (PAD) are both caused by the progressive narrowing of the blood vessels most often caused by atherosclerosis, the collection of plaque or a fatty substance along the inner lining of the artery wall. Over time, this substance hardens and thickens, which can cause an occlusion in the artery, completely or partially restricting flow through the artery. Blood circulation to the arms, legs, stomach and kidneys brain and heart may be reduced, increasing the risk for stroke and heart disease.

Interventional treatments for CAD and PAD may include endarterectomy and/or atherectomy. Endarterectomy is surgical removal of plaque from the blocked artery to restore or improve blood flow. Endovascular therapies such as atherectomy are typically minimally invasive techniques that open or widen arteries that have become narrowed or blocked. Other treatments may include angioplasty to open the artery. For example, a balloon angioplasty typically involves insertion of a catheter into a leg or arm artery and positioning the catheter such that the balloon resides within the blockage. The balloon, connected to the catheter, is expanded to open the artery. Surgeons may then place a wire mesh tube, called a stent, at the area of blockage to keep the artery open.

The ease of performing of such minimally invasive techniques can be enhanced through the use of on-board imaging, such as optical coherence tomography (“OCT”) imaging. Obtaining clear OCT images from a blood vessel, however, can be difficult due to blood in the vessel obstructing the images. One method for improving the clarity of OCT images taken in a blood vessel is to flush the blood away with a saline, such as through a channel in the imaging device or catheter. However, such flushing can be cumbersome, often requiring continuous flushing of greater than 10 cc/sec in order to image. Further, flushing can risk damaging the vessel if too much flush pressure is applied. Accordingly, a mechanism for obtaining clear images from a blood vessel without the need for consistent flushing is desired.

### SUMMARY OF THE DISCLOSURE

An occlusion sheath having an expandable element or balloon is described herein. The central lumen of the occlusion sheath can be large enough to hold a catheter having both imaging and cutting capabilities. Further, the outer diameter of the occlusion sheath can be small enough to fit into a blood vessel, such as a peripheral artery. The balloon can be expanded against a vessel wall to occlude or limit blood flow in the vessel. A catheter, such as a catheter including imaging, e.g., OCT imaging, and cutting capabilities, can be passed through a central lumen of the occlusion sheath. Because blood flow is occluded or limited by the balloon, the catheter can collect images that are not obscured by blood or resolve the obstruction by flushing the blood down the artery with a small amount of fluid.

In general, in one embodiment, an occlusion sheath includes an outer elongate member, an inner elongate member extends within the outer elongate member and a central lumen extends within the inner elongate member. The central lumen has a diameter of greater than 0.055 inches and is configured to house or accommodate a catheter. An occlusion balloon is attached to the outer elongate member and is configured to expand to occlude a vessel.

This and other embodiments can include one or more of the following features. A proximal end of the balloon can be sealed against the outer elongate member and a distal end of the balloon can be sealed against the outer elongate member or the inner elongate member. An inflation lumen can be formed between the outer elongate member and the inner elongate member. The inflation lumen can be in communication with the balloon. The occlusion sheath can further include a hole through the outer elongate member between the proximal end of the balloon and the distal end of the balloon to allow communication between the inflation lumen and the balloon. The hole can be positioned closer to a proximal end of the balloon than a distal end of the balloon. The sheath can further include a tapered tip. The tapered tip can be attached to the outer elongate member with a lap joint. The tapered tip can be radiopaque. An outer diameter of the balloon when fully inflated and unconstrained can be 10-20 mm. An outer diameter of the outer elongate tube can be less than 0.165 inches in diameter. The occlusion sheath can further include a radiopaque marker configured to indicate a position of the balloon. The occlusion sheath can further include a hub attached to the inner and outer elongate members. The hub can include a first port for passage of the catheter and a second port for passage of air or fluid to inflate the balloon. The catheter can be an atherectomy catheter. The catheter can be an imaging catheter.

In general, in one embodiment, a method of performing atherectomy within a vessel includes: (1) inserting an occlusion sheath into a vessel; (2) inserting an atherectomy catheter having an imaging sensor and a cutter through a central lumen of the occlusion sheath; (3) inflating a balloon of the occlusion sheath to occlude the vessel; (4) imaging the vessel with the imaging sensor while the vessel is occluded; and (5) cutting tissue inside the vessel with the cutter.

This and other embodiments can include one or more of the following features. Inflating the balloon can include injecting fluid or air into an annular space between an inner and outer elongate member of the occlusion sheath such that the fluid or air flows down the annular space and into the balloon. Imaging can include imaging with optical coherence tomography. The method can further include placing an introducer through the central lumen of the occlusion sheath prior to inserting the occlusion sheath such that a distal taper of the inserter extends out a distal end of the occlusion sheath. Cutting the tissue can include cutting tissue while the vessel is occluded. The balloon can be inflated for 20-30 minutes. The method can further include deflating the balloon after cutting to resume blood flow through the vessel and then repeating the inflating, imaging, and cutting steps.

In general, in one embodiment, an occlusion sheath includes an elongate member, a central lumen within the elongate member configured to house or accommodate a catheter, an occlusion balloon attached to the elongate member and configured to expand to occlude a vessel, and a sealing mechanism within the central lumen and distal to the occlusion balloon. The sealing mechanism is configured to seal against the catheter when the catheter is within the central lumen such that a balloon inflation lumen is formed between an outer surface of the catheter and an inner surface of the elongate member.

This and other embodiments can include one or more of the following features. The sealing mechanism can be an annular lip. The catheter can be an atherectomy catheter. The catheter can be an imaging catheter.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an exemplary occlusion sheath and hub.

FIGS. 2A and 2B show the distal end of the occlusion sheath of FIG. 1.

FIG. 3 shows the hub of FIG. 1.

FIG. 4 shows exemplary use of the occlusion sheath in a blood vessel.

FIG. 5 shows another embodiment of an exemplary occlusion sheath and hub.

FIG. 6 shows the distal end of the occlusion sheath of FIG. 5.

FIG. 7 shows the hub of FIG. 5.

FIGS. 8A and 8B show another embodiment of an exemplary occlusion sheath and hub.

FIG. 9 shows an exemplary introducer inside an occlusion sheath.

FIGS. 10A and 10B show the occlusion sheath and hub of FIG. 1 with exemplary dimensions.

### DETAILED DESCRIPTION

An occlusion sheath having an expandable element or balloon is described herein. The occlusion sheath is configured to occlude or reduce blood flow to a vessel while providing a central lumen to allow a catheter, such as an imaging catheter and/or an atherectomy catheter, to travel therethrough.

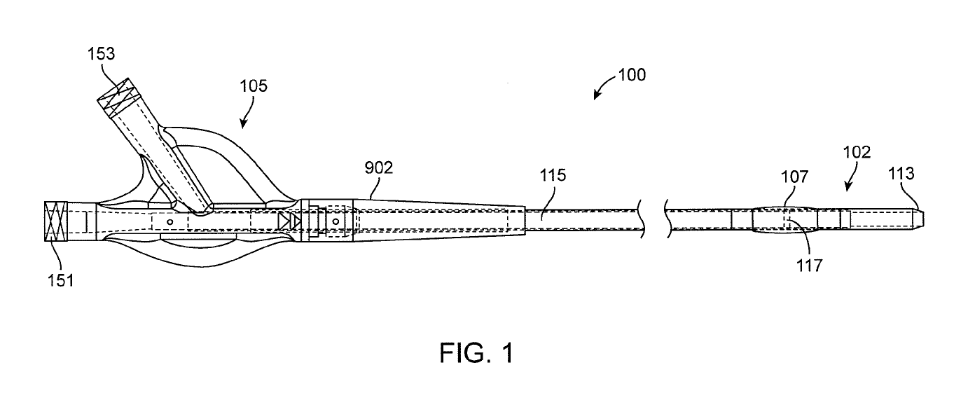

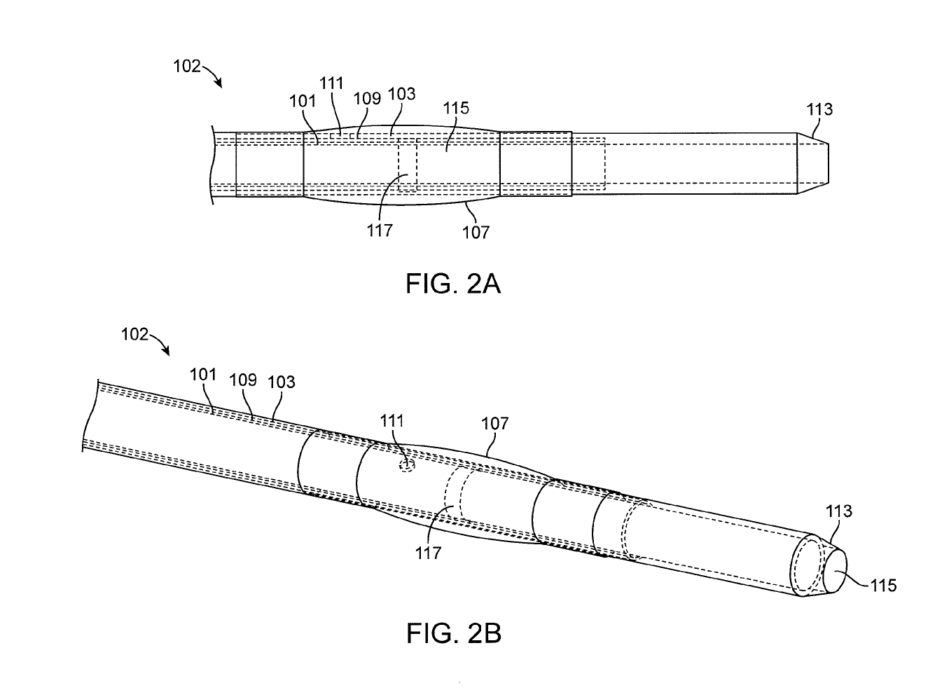

Referring to FIGS. 1-2B, an occlusion sheath 100 includes an inner elongate tube 101, an outer elongate tube 103, and a balloon 107 attached to and around the circumference of the outer elongate tube 103. The balloon 107 is sealed at both ends to the outer elongate tube 103. In other embodiments, the balloon 107 is sealed to the outer tube at the proximal end and the inner tube at the distal end (i.e., the outer tube 103 can end within the balloon 107). The inner elongate tube 101 is coaxial with the outer elongate tube 103 such that an annular space 109 is formed therebetween. The inner elongate tube 101 and outer elongate tube 103 are sealed or fused together near the distal end 102 of the occlusion sheath, thereby also sealing off the distal end of the annular space 109. A central lumen 115 extends axially within the inner elongate tube 101.

At the distal end 102 of the occlusion sheath 100, the outer elongate tube 103 includes one or more holes 111 arranged therearound. The holes 111 are located only between the sealed ends of the balloon 107. The holes 111 can provide a through-passage from the annular space 109 to the interior of the balloon 107. Thus, when fluid or air is passed into the annular space 109 from a proximal end of the sheath 100, it can travel through the holes 111 and into the balloon 107 to inflate the balloon 107. If there is more than one hole 111, the holes 111 can be evenly spaced around the circumference, such as 180 degrees apart if there are two holes 111. In some embodiments, the holes 111 can be located closer to the proximal end of the balloon 107 than the distal end of the balloon 107 to help with de-airing (i.e., the process of removing air and replacing it with saline and contrast). Having the outer elongate tube 103 extend substantially all of the length of the inner elongate tube 101 and/or part of all of the length of the balloon 107 (and thus using holes 111 for inflation) can advantageously prevent formation of a kink point in the sheath and can increase the pushability of the sheath.

The distal end 102 of the occlusion sheath 100 can further include a tapered tip 113 configured to allow for easier transport of the sheath through tortuous anatomy. The tapered tip 113 can be attached to the inner or outer elongate tubes 101, 103 with a lap joint, thereby improving the integrity of the sheath 100. In some embodiments, the tapered tip 113 can be radiopaque and/or include radiopaque elements or compounds. For example, the tip 113 can be made with polyether block amide that is loaded with barium sulfate, platinum, gold, silver, iron, bismuth, or tungsten. By making the tip 113 radiopaque, the tip 113 can advantageously be viewed under fluoroscopy, thereby providing better guidance of the distal end of the sheath 100.

The balloon 107 can be a semicompliant or compliant balloon, thereby advantageously allowing the balloon 107 to expand and conform to the inner diameter of the vessel in which it is inserted. For example, the balloon 107 can be made of polyurethane, silicone, Pebax®, or latex. In some embodiments, the outer diameter of the balloon when fully inflated and unconstrained is 4-20 mm, such as 10-20 mm, such as 12.5 mm, and the working length of the balloon (i.e., the segment of the balloon that extends to the maximum diameter) when fully inflated and unconstrained is approximately 1-15 mm, such as 12 mm long.

The central lumen 115 can be configured to allow a medical catheter, such as an imaging and/or atherectomy catheter, to pass therethrough. Accordingly, the central lumen can have a diameter of greater than 0.055 inches, greater than 0.058 inches, or greater than 0.065 inches. For example, the central lumen can have a diameter of between 0.055 inches and 0.120 inches, such as approximately 0.102 inches, 0.106 inches, or 0.115 inches. Further, the outer diameter of the occlusion sheath 100, i.e., the outer diameter of the outer elongate tube 103, can be less than 0.165 inches in diameter, such as between 0.100 inches and 0.165 inches, e.g., 0.140 inches, allowing the sheath 100 to fit into peripheral arteries, such as the superficial femoral artery. In one embodiment, an outer diameter of the sheath 100 is 11 French while a diameter of the central lumen is 8 French. In another embodiment, an outer diameter of the sheath is 10.5 French while a diameter of the central lumen is 7 French.

As shown in FIGS. 1-2B, the occlusion sheath 100 can further include a radiopaque marker 117 adhered to the inner elongate tube 101. The radiopaque marker 117 can be configured to indicate the position of the balloon 107 under fluoroscopy. For example, the marker 117 can be directly in the center of the balloon and/or at the proximal or distal ends of the balloon or the balloon's working length. The radiopaque marker 117 can be made, for example, of platinum-iradium.

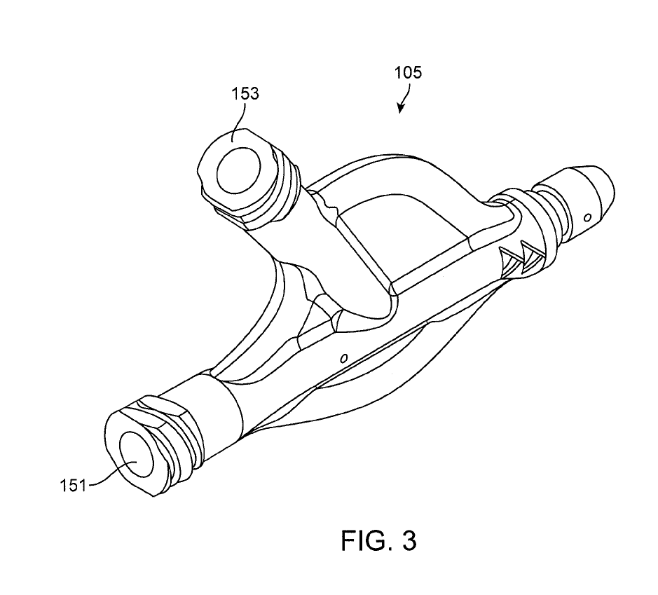

As shown in FIGS. 1 and 3, the occlusion sheath 100 can further include a hub 105 attached to the inner and outer elongate tubes 101, 103 through a tapered strain relief 902. The hub 105 can include first a luer port 151 for passage of a catheter and attachment of a rotating hemostatic valve (RHV) for flushing. The hub 105 can also include a second luer port 153 for attachment of a syringe or other mechanism for providing fluid/air to the annular lumen 109 (see FIGS. 2A and 2B) to inflate the balloon 107. The inner elongate tube 101 can be attached to the hub 105 at a position proximal to luer port 153 while the outer elongate tube 103 can be attached to the hub 105 at a position distal to the luer port 153. As a result, the luer port 153 can connect to the space between the two tubes 101, 103.

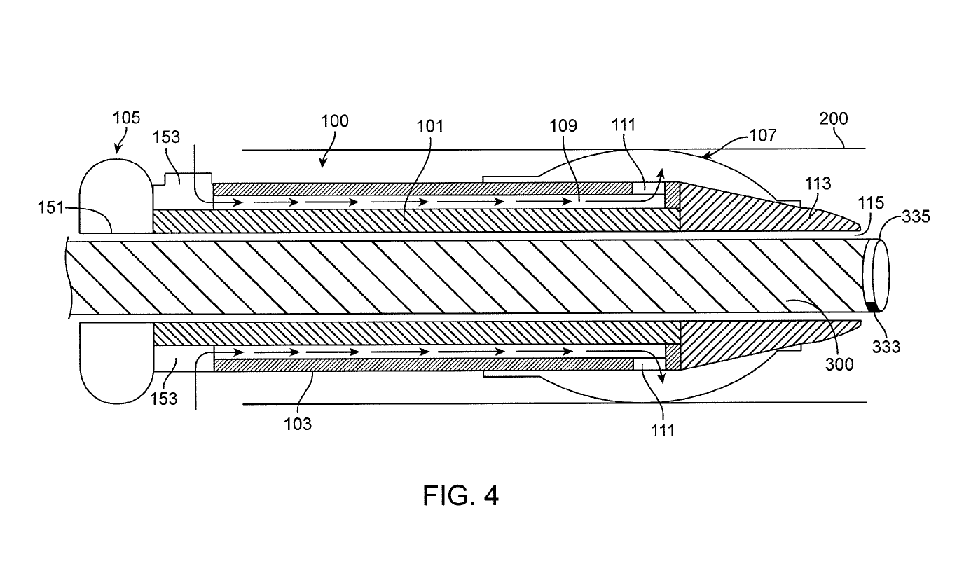

Referring to FIG. 4, in use, the occlusion sheath 100 can be inserted into a blood vessel 200. An imaging catheter 300, such as an atherectomy catheter with an imaging sensor 333 and a cutter 335, can be inserted through the central lumen 115. Exemplary atherectomy catheters are described in U.S. patent application Ser. No. 12/829,277, filed Jul. 1, 2010, titled “ATHERECTOMY CATHETER WITH LATERALLY-DISPLACEABLE TIP,” now U.S. Patent Application Publication No. 2011/0004107, U.S. patent application Ser. No. 13/175,232, filed Jul. 1, 2011, titled “ATHERECTOMY CATHETERS WITH LONGITUDINALLY DISPLACEABLE DRIVE SHAFTS,” now U.S. Patent Application Publication No. 2012/0046679, U.S. patent application Ser. No. 13/654,357, filed Oct. 17, 2012, titled “ATHERECTOMY CATHETERS AND NON-CONTACT ACTUATION MECHANISM FOR CATHETERS,” now U.S. Patent Application Publication No. 2013/0096589, International Patent Application No. PCT/US2013/031901, filed Mar. 15, 2013, titled “ATHERECTOMY CATHETERS WITH IMAGING,” now published as WO 2013/172970, and International Patent Application No. PCT/US2013/032494, filed Mar. 15, 2013, titled “BALLOON ATHERECTOMY CATHETERS WITH IMAGING,” now published as WO 2014/039099, the entireties of which are incorporated by reference herein. In some embodiments, before and during insertion of the catheter 300, blood can flow around the occlusion sheath 100, but not through the central lumen 115 (as there can be a seal between the access site and the lumen 115). In other embodiments, blood can flow both around the occlusion sheath 100 and through the central lumen 115 before or during insertion. Once the sheath 100 is inserted, fluid or air can then be injected through the hub 105 into the annular space 109 between the inner tube 101 and outer tube 103 to inflate the balloon 107. The balloon 107 can be inflated until it touches the wall of the vessel 200, partially or fully sealing off the vessel 200 to limit or eliminate blood flow therethrough. Once blood flow is limited, an image, such as an OCT image, can be taken with the imaging sensor 333. Because blood flow is limited, clear images can be obtained. In some embodiments, some minimal flushing can be performed through the lumen 115 (i.e., such as through the annular space between the outer diameter of the catheter 300 and the inner diameter of the lumen 115) or through the catheter 300 in order to clear out blood that occurred before occlusion, that was trapped during occlusion, or that leaked around the edges of the balloon. In some embodiments, contrast solution can also be injected through the lumen 115 or catheter 300 to improve imaging.

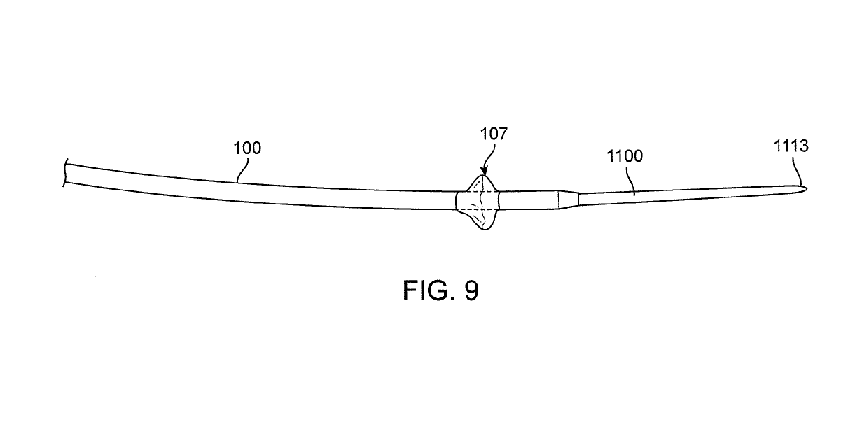

Referring to FIG. 9, an introducer 1100 having a distal taper 1113 can be used to insert the sheath 100. The introducer 1100 can extend through the central lumen 115 and extend past the tip 113 during insertion of the sheath 100. After the sheath 100 is in place, the introducer 1100 can be removed. The introducer 1100 can advantageously create a smooth transition and more pointed end for insertion of the sheath 100 into tortuous anatomy. In some embodiments, the introducer 1100 can allow for placement or exchange of a guidewire therethrough.

Advantageously, the sheath 100 can act as the primary sheath for a catheter, such as an atherectomy catheter. For example, the catheter can be stiff enough to provide support for the catheter inserted therethrough while flexible enough and kink-resistant to curve around tortuous anatomy. The outer and inner elongated tubes 101, 103 can be constructed of polymers reinforced with coiled or braided wire to improve pushability or kink resistance. These wires could be flat or round and made with stainless steel, tungsten, nitinol, or chrome alloys. The polymers could include Pebax, Nylon, HDPE, LDPE, polyurethane or a blend of a plurality of these polymers. The catheter can further be hydrophilically coated to assist in smoothly transferring the sheath 100 through the vessel. The luer lock 151 connection to an RHV can advantageously eliminate bleed-back around the introducer, the catheter, or a guidewire.

The cutter 335 can be used to cut tissue and images can be taken while the sheath 100 is in place. The balloon 107 can advantageously be inflated and deflated multiple times. For example, during an atherectomy procedure, the balloon can be inflated, one or two cuts can be made over a period of, for example, up to 30 minutes, such as up to 20 minutes, and the balloon can be deflated, thereby resuming the blood flow. The process can be repeated until the atherectomy procedure is completed. Likewise, the balloon 107 can be deflated to move the sheath 100.

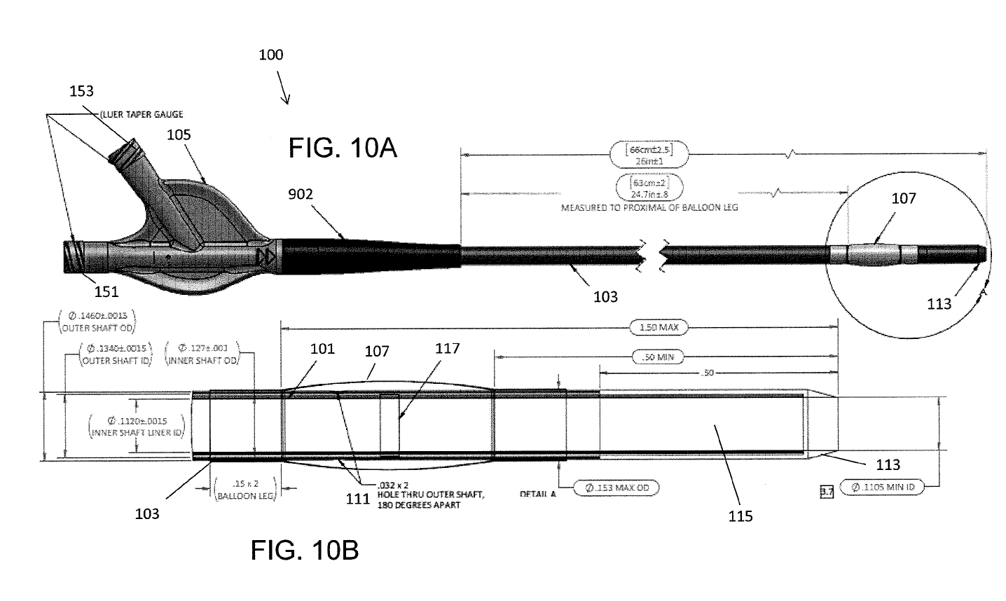

Exemplary dimensions (in inches except where otherwise noted) for an occlusion sheath 100 are shown in FIGS. 10A and 10B. Further, in one exemplary embodiment, the inner elongate tube 101 is made of three layers, including (from inside out): an HDPE liner, a stainless steel coil of 0.0025″ by 0.010″ and 1.7× pitch, and a Pebax® jacket. In one exemplary embodiment, the outer sheath 103 can also be made of three layers, including (from inside out): a Pebax® liner, a stainless steel coil of 0.001″ by 0.005″ and 2× pitch, and a Pellethane® jacket.

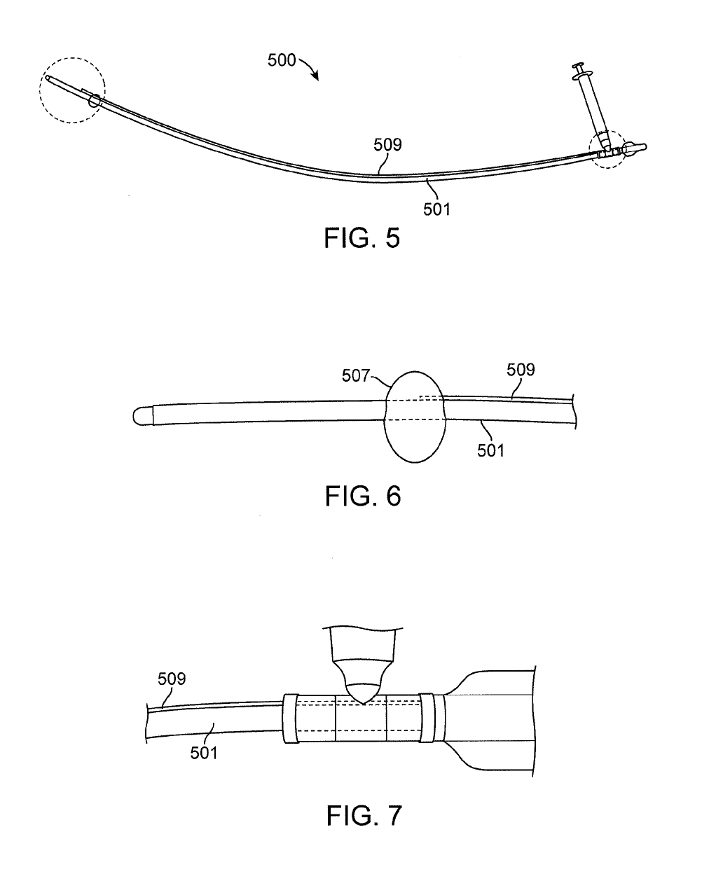

Other embodiments of occlusion sheaths having some or all of the features above are possible. For example, referring to FIGS. 5-7, an occlusion sheath 500 can include a tubular inflation lumen 509 that runs down the side of a single elongate tube 501. The occlusion sheath 500 can otherwise include features, such as a balloon 507 around the elongate tube 501, that are similar to the occlusion sheath 100.

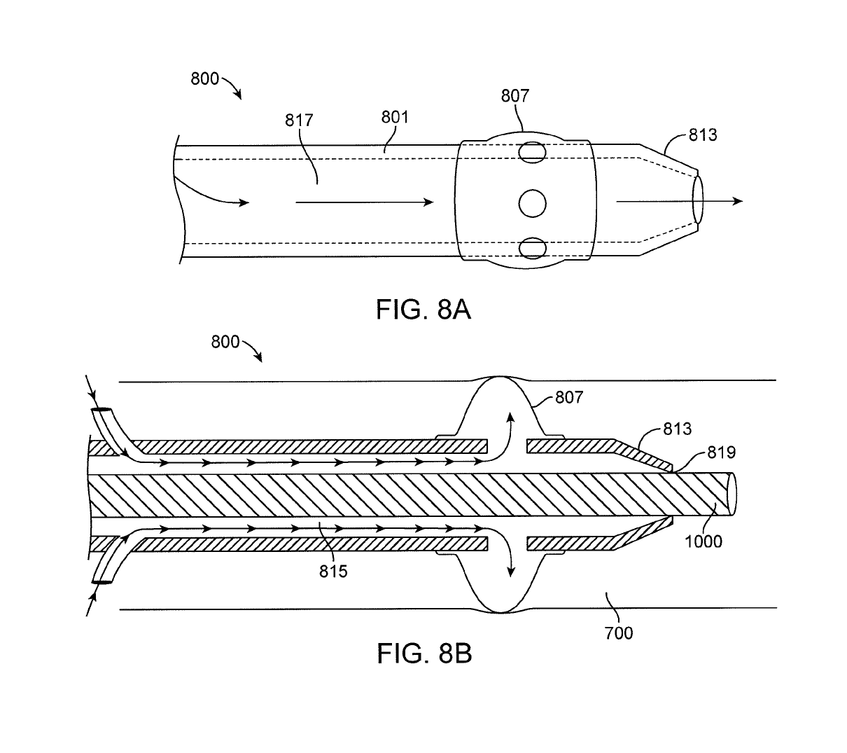

As another example, referring to FIGS. 8A-8B, an occlusion sheath 800 can include a shaft 801 (e.g., a single shaft) and a soft tip 813. The soft tip 813 includes a sealing feature distal of the balloon 807, such as an inner lip 819, configured to create a seal between itself and a catheter inserted through the central lumen 815 of the shaft 801. In some embodiments, rather than being in the tip 813, the seal 819 can be positioned proximal of the tip 813, but distal to a proximal end of the balloon 807, such as under the balloon 807 or at the distal edge of the balloon 807. The lip 819 can be made of an elastic polymer such as silicone, latex, urethane, or Pebax®. The shape of the lip can be configured to create a seal between both cylindrical and irregularly shaped catheter shafts. These shapes can include a circular lip with a reduced diameter, such that inserting the catheter stretches the lip around the catheter shaft. Further, the lip 819 can have a duckbill or clown collared shape, which similarly can be sized to stretch around the catheter shaft. Similar to sheath 100, the sheath 800 can be inserted into a vessel 700. When a catheter 1000 is inserted into the central lumen 815, it creates a seal with the lip 819, thereby allowing air or fluid inserted into the hub 501 to fill the balloon 807, causing the balloon to expand to occlude the vessel 700. Because the occlusion sheath 800 has only one lumen therethrough, the total wall of the sheath can advantageously be much smaller than in other embodiments because this design eliminate a second elongated tube and an inflation lumen. For example, the wall could be between 0.005 and 0.015 inches.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. An occlusion sheath, comprising:
an outer elongate member;
an inner elongate member extending within the outer elongate member;
a central lumen within the inner elongate member, the central lumen having a diameter of greater than 0.055 inches and configured to accommodate a catheter; and
an occlusion balloon attached to the outer elongate member and configured to expand to occlude or reduce the flow through a vessel.

2. The occlusion sheath of claim 1, wherein a proximal end of the balloon is sealed against the outer elongate member, and wherein a distal end of the balloon is sealed against the outer elongate member or the inner elongate member.

3. The occlusion sheath of claim 1, wherein an annular inflation lumen is formed between the outer elongate member and the inner elongate member, the inflation lumen in communication with the balloon.

4. The occlusion sheath of claim 3, further comprising a hole through the outer elongate member between the proximal end of the balloon and the distal end of the balloon to allow communication between the inflation lumen and the balloon.

5. The occlusion sheath of claim 4, wherein the hole is positioned closer to a proximal end of the balloon than a distal end of the balloon.

6. The occlusion sheath of claim 1, wherein the sheath further comprises a tapered tip.

7. The occlusion sheath of claim 6, wherein the tapered tip is attached to the outer elongate member with a lap joint.

8. The occlusion sheath of claim 6, wherein the tapered tip is radiopaque.

9. The occlusion sheath of claim 1, wherein an outer diameter of the balloon when fully inflated and unconstrained is 4-20 mm.

10. The occlusion sheath of claim 1, wherein an outer diameter of the outer elongate tube is less than 0.165 inches in diameter.

11. The occlusion sheath of claim 1, further comprising a radiopaque marker configured to indicate a position of the balloon.

12. The occlusion sheath of claim 1, further comprising a hub attached to the inner and outer elongate members, the hub including a first port for passage of the catheter and a second port for passage of air or fluid to inflate the balloon.

13. The occlusion sheath of claim 1, wherein the catheter is an atherectomy catheter.

14. The occlusion sheath of claim 1, wherein the catheter is an imaging catheter.

15. A method of performing atherectomy within a vessel, comprising:
inserting an occlusion sheath into a vessel;
inserting an atherectomy catheter having an imaging sensor and a cutter through a central lumen of the occlusion sheath;
inflating a balloon of the occlusion sheath to reduce the flow through the vessel;
imaging the vessel with the imaging sensor while the vessel flow is reduced; and
cutting tissue inside the vessel with the cutter.

16. The method of claim 15, wherein inflating the balloon comprises injecting fluid or air into an annular space between an inner and outer elongate member of the occlusion sheath such that the fluid or air flows down the annular space and into the balloon.

17. The method of claim 15, wherein imaging comprises imaging with optical coherence tomography.

18. The method of claim 15, further comprising placing an introducer through the central lumen of the occlusion sheath prior to inserting the occlusion sheath such that a distal taper of the inserter extends out a distal end of the occlusion sheath.

19. The method of claim 15, wherein cutting the tissue comprises cutting tissue while the vessel is occluded.

20. The method of claim 15, wherein the balloon is inflated for 20-30 minutes.

21. The method of claim 15, further comprising deflating the balloon after cutting to resume blood flow through the vessel and then repeating the inflating, imaging, and cutting steps.

22. An occlusion sheath, comprising:
an elongate member;
a central lumen within the elongate member configured to accommodate a catheter;
an occlusion balloon attached to the elongate member and configured to expand to occlude a vessel; and
a sealing mechanism within the central lumen and distal to a proximal end of the occlusion balloon, the sealing mechanism configured to seal against the catheter when the catheter is within the central lumen such that a balloon inflation lumen is formed between an outer surface of the catheter and an inner surface of the elongate member.

23. The occlusion sheath of claim 22, wherein the sealing mechanism is an annular lip.

24. The occlusion sheath of claim 22, wherein the catheter is an atherectomy catheter.

25. The occlusion sheath of claim 22, wherein the catheter is an imaging catheter.
